(19) United States
(12) Patent Application Publication
(10) Pub. No.: US 2004/0039189 A1
(43) Pub. Date: Feb. 26, 2004

(54) Novel amidite derivates for synthesising polymers on surfaces

(76) Inventors: Ramon Guimil, Heidelberg (DE); Matthias Scheffler, Hirschberg/Leutershausen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/362,503

(22) PCT Filed: Aug. 24, 2001
(86) PCT No.: PCT/EP01/09812

(30) Foreign Application Priority Data
Aug. 24, 2000 (DE) 100 41 539.3

(51) Int. Cl. C07F 9/06; C07F 9/547; C07H 19/04

(52) U.S. Cl. 536/26.1; 546/21; 548/412

(57) ABSTRACT

The invention relates to novel amidite derivatives and their use as linker units for synthesizing polymers, especially biopolymers such as nucleic acids, peptides and saccharides, on the surface of solid supports. Use of the linker derivatives of the invention makes it possible to regenerate the surfaces without damage.

### DESCRIPTION

The invention relates to novel amidite derivatives and their use as linker units for synthesizing polymers, especially biopolymers such as nucleic acids, peptides and saccharides, on the surface of solid supports. Use of the linker derivatives of the invention makes it possible to regenerate the surfaces without damage.

Since the introduction of the synthesis of biopolymers on solid surfaces by Merryfield in 1965, a large number of publications about this technique has appeared. A more recent development in this area is the preparation of so-called biopolymer arrays, where a large number of different biopolymers such as, for example, nucleic acids or peptides are immobilized in defined areas on a support.

In the synthesis of biopolymers on a solid phase there is generally use of a spacer between the support and the actual biopolymer. The use of a spacer has the advantage that the biopolymer is further removed from the surface of the solid support, so that the influences thereof are diminished, so that the immobilized biopolymer can undergo quasi-homogeneous reactions. The nature of the spacer, its length, polarity and other physicochemical properties accordingly have a crucial influence on the coupling yield in the synthesis of polymer on the support, and thus on the quality of the polymer and on its later use.

The strategies generally used to assemble spacers are based on polycondensation of, in the widest sense, amino acid monomers or cyanoethyl- or otherwise protected phosphoramidites or H-phosphonates. The compounds normally used in these cases have the following general structure:

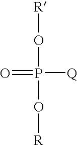

in which R′ is a protected linker group, R is the precursor synthon or the functional group of a solid phase, and Q is H or an organic protective group such as cyanoethoxy or methoxy, which plays no part in the anchoring on the surface.

A considerable disadvantage of the spacer molecules disclosed to date are the negative charges which occur during the elimination of the protective groups on the phosphate groups. These additional potential coupling centers prevent possible recycling by suitable chemical or enzymatic procedures of the spacers.

The object on which the invention is based was thus to provide novel spacer units for synthesizing polymers on solid supports. This object is achieved by a compound of the general formula (I)

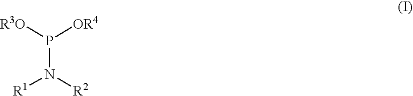

in which R¹ and R² are each independently a C₁-C₁₀ hydrocarbon radical, e.g. a C₁-C₆ alkyl radical or a C₃-C₄ cycloalkyl radical, or are connected together, e.g. to result in a 5- or 6-membered ring, and R³ and R⁴ are each independently a protected linker group of the general formula (II):

—L—(X)ₙ

in which L is a linker, X is a protective group and n is an integer from 1-3.

R¹ and R² in the compounds (I) are preferably in each case methyl, ethyl or i-propyl radicals or they form together a morpholine radical. It is, of course, also possible to use other alkyl or cycloalkyl radicals.

The linker group (II) generally comprises linear or branched aliphatic, olefinic or/and aromatic hydrocarbon groups which are optionally substituted by heteroatoms. It preferably comprises an alkylene chain in which, where appropriate, one or more CH₂ groups can be replaced by heteroatoms such as O, S or NH. The chain length of the linker is preferably 1-100 atoms, preferably 10-45 atoms and particularly preferably 10-25 atoms. The chain may furthermore comprise one or more branches, it being possible for a linker group preferably to comprise up to three branches. The branches may be introduced into the linker group for example through ω-bis- or trishydroxy compounds such as, for example, tris(hydroxymethyl)aminomethane. In a particularly preferred embodiment, the linker group (II) comprises a structure selected from (a) (CH₂)ₙ—X in which n=1-12, preferably 3-6, or (b) [(CH²)ᵣO]ₛ—X in which r=1-4, preferably 2-3, and s=1-100, preferably 3-9.

Located at the end or at the ends of the linker group there are one or more protective groups X which may be selected from protective groups known for the solid-phase synthesis of nucleic acids or peptides. X is preferably a protective group which can be eliminated from the linker by chemical or enzymatic reactions, the elimination releasing a reactive group, e.g. a hydroxyl or amino group. Examples of suitable protective groups are acid-labile protective groups such as, for example, dimethoxytrityl (DMT), MMT, pixyl, Fpmp, base-labile protective groups such as, for example, benzyl, benzoyl, isobutyryl, phenoxyacetyl, levulinyl etc., oxidation- or/and reduction-labile protective groups, photolabile protective groups such as, for example, NVOC, NPPOC, protective groups which can be eliminated catalytically, e.g. by Pd, such as, for example, allyl, AOC, and protective groups which can be eliminated by fluoride, such as, for example, TMS and derivatives thereof, e.g. TBDMS.

The compounds of the invention are outstandingly suitable as spacers or spacer units for synthesizing polymers on solid supports. It is possible to employ one or more molecules of compounds (I) for synthesizing a polymer molecule. This normally entails first the compounds (I) being coupled to the support to assemble the spacer, and subsequently the polymer being synthesized on the spacer with use of suitable synthons. Suitable solid phases are inorganic or organic supports, e.g. functionalized controlled pore glass (CPG), other glasses such as Foturan, Pyrex or usual sodalime glasses, metallic supports such as, for example, silicon, or organic resins such as, for example, Tentagel. It is particularly preferred for the support to be a chip employed for synthesizing polymer arrays.

One aspect of the present invention is thus also a support for solid-phase synthesis of the general formula (Ia)

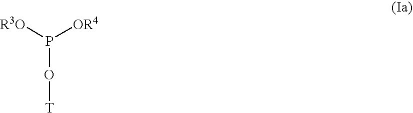

in which T is a solid support as previously indicated, and R³ and R⁴ are as previously defined. The support (Ia) is prepared by coupling a compound (I) to a reactive group of the support, e.g. a hydroxyl group, with elimination of the —NR¹NR² group. It is possible where appropriate to oxidize the support (Ia) for example with molecular I₂, in which case the P atom is converted from oxidation state III into oxidation state V.

At least one of the protective groups X on the linkers R³ and R⁴ can be eliminated. It is possible for one or more polymers to be synthesized on the reactive groups which have been released after elimination of the protective groups X, and these polymers can be selected from, for example, nucleic acids such as DNA or RNA, nucleic acid analogs such as, for example, PNA or LNA, peptides or saccharides.

The compounds (I) of the invention are distinguished by requiring no P(V) protective group and carrying no negative charge after deblocking and, where appropriate, oxidation to the phosphate. It is possible in this way for the compounds of the invention to be employed in cooperation with other synthons, e.g. trifunctional sugar or nucleotide units which are provided with orthogonal protective groups, for assembling polymers and for damage-free recycling of the surfaces without the occurrence of charges which would interfere with subsequent syntheses.

This entails firstly the compound (I) of the invention being coupled to a reactive group, e.g. a hydroxyl group on the solid support. The protective group X is subsequently cleaved, the protective group X being orthogonal to a protective group Y on the polymer synthon. After completion of the polymer synthesis and, where appropriate, deblocking of the resulting polymers, the protective group Y, which is orthogonal to the synthetic conditions used previously, is eliminated. An example of such a synthon having the general formula (III) is as follows:

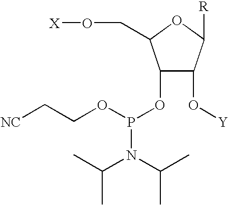

in which X and Y are mutually orthogonal protective groups, where X is, for example, an acid- or/and photolabile protective group, and Y is a protective group which can be eliminated by catalysis, and R is a nucleobase or a fluorophore, a chromophore or another labeling group. Elimination of Y in a basic medium is followed by attack of the 2′-hydroxyl group on the 3′-phosphate to form a cyclic phosphate, which is equivalent to hydrolysis and leads to restoration of the original hydroxyl group.

It is possible on use of an RNA part-segment as probe “socket” for chemical regeneration of the reaction support to take place. In this case, firstly the synthesis is carried out using 2′-OH-protected phosphitamide units. After hybridization, the protective group of the RNA part-segment is eliminated, resulting in a free 2′-OH group. It is then possible in a following chemical reaction step using periodate or other oxidizing agents to cleave the ribose sugar and remove the probe from the reaction support by β elimination.

The compound Ia of the invention can also be employed by suitable biochemical approaches without coupling in a specific molecule for damage-free recycling of the surfaces. In this case, the polymer or oligomer probes linked to the reaction support are cleaved with a DNA- or RNA-degrading enzyme or a peptide-cleaving enzyme, resulting in partial or complete degradation of the probes. It is subsequently possible for the reaction support to be used anew for synthesizing new probes.

Suitable enzymes are nucleases such as exonucleases or endonucleases, which respectively attack a nucleic acid strand from the ends of or within the probe strand, and leave behind nucleotides or nucleosides as cleavage products. In the case of RNA, it is possible to use RNAses (such as RNAse H etc.) which, when an RNA-DNA double strand has formed, selectively cut up the RNA part, with cleavage of the entire probe in the case of RNA probes, and of the RNA segment in the case of RNA part-segments as intended break point. Regeneration of a reaction support with DNA probes can likewise be achieved by using DNAses (DNAse I, DNAse II, etc.), by which means it is possible to degrade both single-stranded and double-stranded DNA.

It is likewise possible to employ peptide-cleaving enzymes to degrade peptide probes or peptide sequence segments as intended break point.

A further advantage of the compounds of the invention is that, because of the branching, a signal amplification takes place because the doping density of the functional groups on the surface is increased. The compounds of the invention are further distinguished in that they make less costly polymer synthesis possible and can be directly integrated into DNA solid-phase synthesis with a simultaneous reduction in the amidite ports required compared with the use of commercially obtainable amidites.

The compounds of the invention are prepared by reacting the monoprotected spacer basic molecules with phosphorus trichloride to give the disubstituted monochloro derivative. The secondary amine is then introduced into the molecule subsequently.

The invention is to be explained further by the following example.

### EXAMPLE 1

### Preparation of Bis(9-O-dimethoxytrityl Triethylene Glycol) [N,N-diisopropyl]Phosphoramidite

1.37 g (10 mmol) of PCl₃ and 5 equivalents of N-ethyldiisopropylamine or pyridine are taken up in absolute ether under protective gas. Monotritylated triethylene glycol dissolved in ether and N-ethyl-diisopropylamine is slowly added dropwise to this cooled solution. After stirring at room temperature for two hours, 3 equivalents of diisopropylamine dissolved in ether are slowly added dropwise to the reaction mixture. The mixture is then left to stir at room temperature for a further 12 h. After concentration, the mixture is taken up in methylene chloride and extracted several times with the usual solutions before the substance is then isolated by chromatography on silica gel.

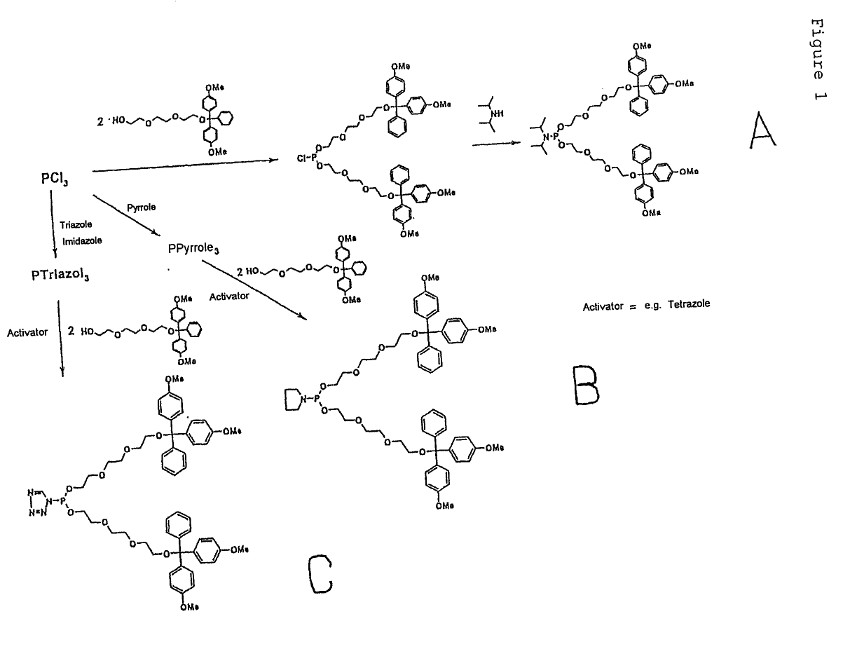

The synthetic scheme is depicted in FIG. 1A.

### EXAMPLE 2

As an alternative to the procedure described in example 1, PCl₃ can also be reacted with heterocyclic nitrogen bases such as pyrrole, triazole or imidazole, and only then be reacted with monotritylated triethylene glycol, where appropriate in the presence of an activator such as, for example, tetrazole.

The corresponding synthetic schemes are depicted in FIG. 1B and 1C.

1. A compound of the general formula (I) 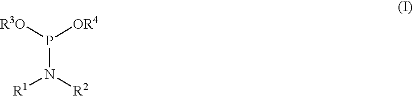
in which r¹ and r² are each independently a C₁-C₁₀ hydrocarbon radical or are connected together to result in a 5- or 6-membered ring, and r³ and r⁴ are each independently a protected linker group of the general formula (ii):
—L—(X)ₙ (II)
in which L is a linker, X is a protective group and n is an integer from 1-3.

2. The compound as claimed in claim 1, characterized in that R¹ and R² are each independently a C₁-C₆ alkyl radical or a C₃-C₄ cycloalkyl radical.

3. The compound as claimed in claim 1 or 2, characterized in that R¹ and R² are each methyl, ethyl or i-propyl or together form a morpholine radical.

4. The compound as claimed in any of claims 1 to 3, characterized in that the linker group (II) comprises linear or branched aliphatic, olefinic or/and aromatic hydrocarbon groups which are optionally partially substituted by heteroatoms.

5. The compound as claimed in any of claims 1 to 4, characterized in that the linker group (II) comprises
(a) a structure —(CH₂)ₘ—X
in which m=1-12 or
(b) a structure —[(CH₂)ᵣO]ₛ—X
in which r=1−4 and s=1−100.

6. The compound as claimed in any of claims 1 to 5, characterized in that X is a protective group selected from protective groups which can be eliminated by chemical or enzymatic reactions.

7. The compound as claimed in claim 6, characterized in that X is an acid-labile protective group.

8. The compound as claimed in claim 6, characterized in that X is a base-labile protective group.

9. The compound as claimed. in claim 6, characterized in that X is an oxidation- or/and reduction-labile protective group.

10. The compound as claimed in claim 6, characterized in that X is a photolabile protective group.

11. The compound as claimed in claim 6, characterized in that X is a protective group which can be eliminated catalytically.

12. The compound as claimed in claim 6, characterized in that X is a protective group which can be eliminated by fluoride.

13. A support for solid-phase synthesis of the general formula (Ia) 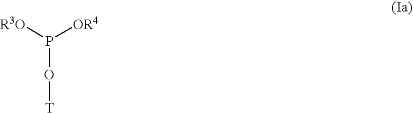
in which T is a solid support and R³ and R⁴ are as defined in any of claims 1 and 4 to 12.

14. The support as claimed in claim 13, characterized in that at least one protective group X in the linker groups R³ and R⁴ is eliminated.

15. The support as claimed in claim 14, characterized in that one or more polymers are synthesized on the reactive groups released after elimination of the protective groups X.

16. The support as claimed in claim 15, characterized in that the polymer is selected from nucleic acids, nucleic acid analogs, peptides and saccharides.

17. A process for synthesizing polymers on a solid support, characterized in that a compound of the formula (I), as defined in claims 1 to 12, is covalently coupled to the solid support, and then a polymer is assembled from synthons on the compound (I).

18. The process as claimed in claim 17, characterized in that the compound (I) is coupled to a hydroxyl group on the solid support.

19. The process as claimed in claim 17 or 18, characterized in that the polymers are selected from nucleic acids, nucleic acid analogs, peptides and saccharides.
